(19) United States
(12) Patent Application Publication
(10) Pub. No.: US 2026/0263116 A1
(43) Pub. Date: Sep. 10, 2026

(54) SPINAL ROD AND SYSTEMS THEREOF

(71) Applicant: SPINAL RESOURCES, INC., Fort Lauderdale, FL (US)

(72) Inventor: Bernard M. Bedor, Fort Lauderdale, FL (US)

(21) Appl. No.: 19/560,512

(22) Filed: Mar. 9, 2026

Related U.S. Application Data
(60) Provisional application No. 63/768,410, filed on Mar. 7, 2025.

(51) Int. Cl. A61B 17/70

(52) U.S. Cl. CPC A61B 17/7004; A61B 17/7031

(57) ABSTRACT

Disclosed are spinal rods, systems, and methods for improving long-term outcomes in spinal fusion surgery. The spinal rods include multiple linear segments of varying diameters connected by tapered segments, such as Bezier zones, that provide smooth transitions and variable stiffness along the rod. The segment lengths, diameters, tapering, and positioning are adapted to a patient's anatomy to distribute forces evenly, reduce stress concentrations, and minimize complications such as stress shielding, Proximal Junctional Kyphosis (PJK), Proximal Junctional Failure (PJF), and Adjacent Segment Disease. A computational system receives preoperative and intraoperative assessment data, including imaging, physician observations, and patient anatomical dimensions, and outputs a patient-specific rod configuration and curvature template with real-time surgical instructions. The system further accounts for bilateral forces across the spinal column to achieve symmetric load transmission.

### CROSS REFERENCE TO RELATED APPLICATIONS

The application claims priority to U.S. Provisional Patent Application No. 63/768,410, filed March 7, 2025, entitled “SPINAL ROD AND METHODS THEREOF,” of which is incorporated herein by reference in its entirety.

### FIELD OF THE INVENTION

The present disclosure relates generally to a spinal rod and, more particularly, to spinal rod systems and methods for determining curvature and implantation of a spinal rod.

### BACKGROUND

Spinal rods are often used as components in spinal fusion surgeries, providing structural support to stabilize the spine and facilitate proper healing. These spinal rods usually comprise a constant diameter across its length or a stepped diameter where the diameter immediately decreases between two constant diameter sections and are typically made from materials such as titanium, cobalt-chromium, and stainless steel, each offering different levels of strength, flexibility, and biocompatibility. While the primary function of the spinal rods is to maintain spinal alignment and promote stabilization of a patient’s anatomy, the mechanical properties of spinal rods significantly influence postoperative outcomes and can cause complications.

One of the key challenges in spinal rod design is achieving an optimal balance between rigidity and flexibility. Excessive stiffness can lead to stress shielding, where the natural load-bearing function of the spine is diminished, potentially resulting in bone weakening and implant-related complications such as loosening or failure. Additionally, rigid constructs have been associated with an increased risk of Proximal Junctional Kyphosis (PJK), a condition characterized by abnormal curvature at the upper segment of the stabilized spine, which may progress to mechanical failure and require revision surgery. On the other hand, spinal rods that lack sufficient rigidity may fail to provide adequate spinal stabilization, leading to inadequate fusion and potential nonunion where the vertebrae do not properly heal together. Moreover, insufficient stiffness can result in excessive micromotion at the fusion site, increasing the risk of implant fatigue, rod breakage, and progressive spinal deformity, ultimately similarly necessitating revision surgery like its rigid counterparts.

There is a need for improved spinal rods, systems, and methods that can maintain spinal realignment while treating, preventing, or minimizing the risk of complications like stress shielding, Proximal Junctional Kyphosis (PJK), Proximal Junctional Failure (PJF), and Adjacent Segment Disease of the lumbar spine or other region. There is a need for one or more (or all) of: variable stiffness constructs; dynamic stabilization techniques including, for example, specific unloading of forces at the Upper Instrumented Vertebra (UIV) proximal segment and specific adjustments to account for and match bilateral forces; patient customization; and in-situ adjustments or adaptability to get to neutral and address current anatomy as well as to prevent future disease state and complications; each and together with the intention to enhance long-term surgical outcomes.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. Any of the described aspects may be isolated or combined with other described aspects without limitation to the same effect as if they had been described separately and in every possible combination explicitly.

Accordingly, there is a need in the art for spinal rods, systems, and methods that can maintain spinal realignment while treating, preventing, or minimizing the risk of complications like stress shielding, Proximal Junctional Kyphosis (PJK), Proximal Junctional Failure (PJF), and Adjacent Segment Disease of the lumbar spine or other region. The disclosed spinal rods, systems, and methods can provide one or more (or all) of: variable stiffness constructs; dynamic stabilization techniques including, for example, specific unloading of forces at the Upper Instrumented Vertebra (UIV) proximal segment and specific adjustments to account for and match bilateral forces; patient customization; and in-situ adjustments or adaptability to get to neutral and address current anatomy as well as to prevent future disease state and complications; each and together with the intention to enhance long-term surgical outcomes.

According to one aspect, the present disclosure provides a spinal rod for spinal surgery. The spinal rod comprises a plurality of linear segments arranged along a longitudinal axis of the spinal rod. The plurality of linear segments comprise at least a first linear segment having a first diameter and a second linear segment having a second diameter different from the first diameter. The spinal rod further comprises at least one tapered segment disposed between adjacent linear segments of the plurality of linear segments, the at least one tapered segment configured to provide a gradual transition in diameter between the adjacent linear segments. The plurality of linear segments and the at least one tapered segment are configured to provide a variable stiffness along the longitudinal axis of the spinal rod. The at least one tapered segment is configured to distribute force across a surface of the spinal rod. A length, a diameter, a tapering, a location, and a number of the plurality of linear segments and the at least one tapered segment are configurable to a patient's anatomy.

In some embodiments, the first linear segment and the second linear segment are adjacent linear segments, and the at least one tapered segment is disposed between the first linear segment and the second linear segment. In some embodiments, the at least one tapered segment comprises a Bezier tapered segment. In some embodiments, a proximal portion of the spinal rod comprises a first material and a distal portion of the spinal rod comprises a second material different from the first material, wherein the first material is more flexible than the second material, and wherein the first material comprises titanium and the second material comprises cobalt-chromium. In some embodiments, the plurality of linear segments comprise at least three linear segments having different lengths, and the at least three linear segments are arranged in a predetermined order along the longitudinal axis based on the patient's anatomy. In some embodiments, the spinal rod has a curvature configured to follow a natural sagittal curvature of the patient. In some embodiments, the spinal rod further comprises at least one curved segment arranged along the longitudinal axis of the spinal rod.

In another aspect, the present disclosure provides a spinal rod system for determining a configuration and placement of a spinal rod in a patient. The system comprises a processor and a non-transitory computer-readable medium storing instructions that, when executed by the processor, cause the system to receive one or more inputs related to a preoperative assessment and an intraoperative assessment of the patient. The one or more inputs comprise at least one of imaging data, physician observations, diagnoses, or anatomical dimensions of the patient. The instructions further cause the system to determine, based on the one or more inputs, a spinal rod configuration comprising a plurality of linear segments and at least one tapered segment configured to provide variable stiffness. The spinal rod configuration is based on the patient's spinal alignment, bone quality, and anatomical dimensions. The instructions further cause the system to generate a curvature template based on the spinal rod configuration and to provide output instructions for at least one of in-situ adjustment or implantation of the spinal rod based on the spinal rod configuration and the curvature template.

In some embodiments, the spinal rod configuration is determined based on stress distribution and load transfer associated with the patient's spine. In some embodiments, the curvature template comprises a failure-state risk percentage. In some embodiments, the one or more inputs further comprise at least one of MRI data, CT data, DEXA scan data, osteotomy angles, sagittal balance, pelvic incidence, or interbody cage angles. In some embodiments, the instructions further cause the system to analyze the one or more inputs using at least one of computational modeling, artificial intelligence, or intraoperative navigation. In some embodiments, the output instructions comprise one or more of rod contouring, rod rotation, sequential compression and distraction, rod translation, rod stiffness modulation, cross-link placement, screw type and placement selection, or rod cutting and end modification. In some embodiments, the instructions further cause the system to determine a first spinal rod configuration for a first side of the patient's spinal column and a second spinal rod configuration for a second side of the patient's spinal column, the first spinal rod configuration and the second spinal rod configuration being configured to provide symmetric load transmission across the spinal column. In some embodiments, the instructions further cause the system to output a plurality of spinal rod configuration options based on the patient's anatomy.

In yet another aspect, the present disclosure provides a method of implanting a spinal rod in a patient. The method comprises performing a preoperative assessment on the patient, performing an intraoperative assessment on the patient, and providing one or more inputs related to the preoperative assessment and the intraoperative assessment into a computational system. The method further comprises analyzing, by the computational system, the one or more inputs to generate output instructions for implantation of the spinal rod. The spinal rod comprises a plurality of linear segments and at least one tapered segment. The method further comprises performing, based on the output instructions, at least one in-situ adjustment to the spinal rod, and implanting the spinal rod in the patient.

In some embodiments, the at least one in-situ adjustment comprises one or more of rod contouring, rod rotation, sequential compression and distraction, rod translation, rod stiffness modulation, cross-link placement, screw type and placement selection, or rod cutting and end modification. In some embodiments, the method further comprises implanting a second spinal rod on an opposite side of the patient's spinal column, wherein the spinal rod and the second spinal rod are configured to provide symmetric load transmission across the spinal column. In some embodiments, the one or more inputs comprise at least one of imaging data, physician observations, diagnoses, or anatomical dimensions of the patient. In some embodiments, the analysis further generates a curvature template comprising a failure-state risk percentage.

The following description and the drawings disclose various illustrative aspects. Some improvements and novel aspects may be expressly identified, while others may be apparent from the description and drawings.

### BRIEF DESCRIPTION OF THE DRAWINGS

The present teachings may be better understood by reference to the following detailed description taken in connection with the following illustrations, in which like reference characters refer to like parts throughout.

FIG. 1 is a perspective view of example first and second stabilizing assemblies, each comprising a spinal rod having a plurality of regions with a different cross-sectional dimension attached to vertebral members according to one or more embodiments.

FIG. 2 is a perspective view of an example spinal rod and a plurality of screws for anchoring a spinal implant to vertebral members.

FIG. 3 is a top view of an example spinal rod comprising three linear regions, each having a different diameter, and tapered transition regions separating the linear regions from each other.

FIG. 4 is an enlarged view of the example transition region encircled by broken lines in FIG. 3.

FIG. 5 shows an example embodiment of a method for determining curvature and placement of a spinal rod in a patient in accordance with aspects disclosed herein.

FIG. 6 shows an example embodiment of a computational system for determining curvature and placement of a spinal rod in a patient in accordance with aspects disclosed herein.

FIG. 7 shows an embodiment of a step of a method for determining curvature and placement of a spinal rod in a patient in accordance with aspects disclosed herein.

FIG. 8 shows an embodiment of a step of a method for determining curvature and placement of a spinal rod in a patient in accordance with aspects disclosed herein.

FIG. 9 shows an embodiment of a step of a method for determining curvature and placement of a spinal rod in a patient in accordance with aspects disclosed herein.

FIG. 10 shows an embodiment of a step of a method for determining curvature and placement of a spinal rod in a patient in accordance with aspects disclosed herein.

FIG. 11 shows an embodiment of a step of a method for determining curvature and placement of a spinal rod in a patient in accordance with aspects disclosed herein.

FIG. 12 shows an embodiment of a step of a method for determining curvature and placement of a spinal rod in a patient in accordance with aspects disclosed herein.

FIG. 13 shows an embodiment of a step of a method for determining curvature and placement of a spinal rod in a patient in accordance with aspects disclosed herein.

FIG. 14 shows an embodiment of a step of a method for determining curvature and placement of a spinal rod in a patient in accordance with aspects disclosed herein.

FIG. 15 shows an embodiment of a step of a method for determining curvature and placement of a spinal rod in a patient in accordance with aspects disclosed herein.

The present teachings may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present teachings, examples of which are illustrated in the accompanying drawings, wherein like numbered aspects refer to a common feature throughout. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Further, as herein disclosed, the terms “substantially,” “about,” and variations thereof describe features that are equal or approximately equal to a value or characteristic, as desired, reflecting tolerances, conversion factors, rounding off, measurement error, acceptable variation thresholds, and the like. For example, unless context or this disclosure suggests otherwise, the term “substantially” includes values or characteristics that are exact or within 15% of exact (or what is stated), for example within 10% of exact, or within 5% of exact. In another example, unless context or this disclosure suggests otherwise, the term “about” includes values within .5 of a degree to 1 degree of exact (or what is stated).

Further, unless context or this disclosure suggests otherwise, descriptions of shapes (e.g., circular, rectangular, triangular, etc.) refer to shapes meeting the definition of such shapes and general representation of such shapes. For instance, a triangular shape or generally triangular shape may include a shape that has three sides and three vertices or a shape that generally represents a triangle, such as a shape having three major sides that may or may not have straight edges, triangular like shapes with rounded vertices, etc.

Disclosed are spinal rods, systems, and methods that can maintain spinal realignment while treating, preventing, or minimizing the risk of complications like stress shielding, Proximal Junctional Kyphosis (PJK), Proximal Junctional Failure (PJF), and Adjacent Segment Disease of the lumbar spine or other region. The disclosed spinal rods, systems, and methods can provide one or more (or all) of: variable stiffness constructs; dynamic stabilization techniques including, for example, specific unloading of forces at the Upper Instrumented Vertebra (UIV) proximal segment and specific adjustments to account for and match bilateral forces; patient customization; and in-situ adjustments or adaptability to get to neutral and address current anatomy as well as to prevent future disease state and complications; each and together with the intention to enhance long-term surgical outcomes.

In an example, the disclosed spinal rods, systems, and methods can be used to manipulate and account for forces at the UIV proximal segment. In an example, the disclosed spinal rods, systems, and methods can be additionally or alternatively be used to treat one or more (or all) of: PJK, PJF, and/or Adjacent Segment Disease of the lumbar spine or other regions. For example, the disclosed spinal rods, systems, and methods may comprise multiple linear segments and multiple different lengths of linear segments. These different lengths of linear segments may be placed in different orders. For example, the linear segment order may be 4.75, 6.0, 5.5 lengths or 4.75, 5.0, 5.5 lengths, etc. (measured in millimeters). It is noted that any lengths, order of lengths, diameters, tapering (e.g., Bezier zones), extent of tapering, location of segments, number of segments, curvature, etc., and combinations thereof, may be used. The lengths, order of lengths, diameters, tapering (e.g., Bezier zones), extent of tapering, location of segments, number of segments, curvature, etc., and combinations thereof, may be determined by or adapted to patient anatomy, for example. The lengths, order of lengths, diameters, tapering (e.g., Bezier zones), extent of tapering, location of segments, number of segments, curvature, etc., and combinations thereof, may contribute to overall construct stiffness reduction and a softer unloading at the UIV and the proximal screws. Bezier zone or Bezier tapered segment refers to a tapered segment whose outer profile (i.e., the change in cross-sectional diameter along the longitudinal axis) is defined by a Bezier curve, a parametric curve determined by a set of control points.

A Bezier curve is a parametric curve defined by a polynomial function of degree n, where n is determined by the number of control points minus one. The taper profile may be defined by a quadratic Bezier curve (three control points), a cubic Bezier curve (four control points), or a higher-order Bezier curve, depending on the complexity of the desired transition. The first and last control points may correspond to the diameters of the two adjacent linear segments (e.g., the first linear segment's diameter and the second linear segment's diameter), and one or more intermediate control points may define the shape, rate, and curvature of the transition between those diameters. The selection of control points, and thus the specific shape of the Bezier taper, may be determined based on the patient's anatomy, bone quality, spinal alignment, and desired stiffness gradient, consistent with the patient-customization process described herein] Unlike a simple linear (conical) taper or a stepped diameter transition (as described above for conventional rods), a Bezier-defined taper provides a smooth, continuously curved transition with tangent continuity at each endpoint, which avoids discontinuities and abrupt changes in cross-section.

More specifically, a Bezier tapered segment, as used herein, refers to a tapered segment whose outer profile, i.e., the change in cross-sectional diameter along the longitudinal axis, is defined by a Bezier curve. A Bezier curve is a parametric curve defined by a set of control points, where the first and last control points correspond to the endpoints of the curve (here, the diameters of the adjacent linear segments) and one or more intermediate control points define the shape and rate of the transition. The Bezier taper may be defined by different orders of Bezier curves, e.g., quadratic (three control points), cubic (four control points), or higher-order curves, and that the selection of curve order and control point positions depends on the desired transition profile and patient anatomy. The Bezier curve defines the outer diameter profile of the tapered segment as a function of position along the longitudinal axis. The first and last control points are set at the diameters of the two adjacent linear segments, ensuring a smooth connection at each junction. The intermediate control points determine the rate and curvature of the diameter transition, for example, whether the taper is more gradual near one end and sharper near the other, or uniformly curved throughout. Unlike a stepped transition (which creates an abrupt discontinuity in cross-section) or a linear/conical taper (which produces a straight-line diameter change), a Bezier taper provides a smooth, continuously curved transition that can be tailored to specific geometric and biomechanical requirements. The smooth curvature of the Bezier profile avoids abrupt changes in cross-section that would create stress concentrations, and that the tangent continuity inherent in Bezier curves ensures that the stiffness transition is gradual rather than discontinuous.

Generally, the forces acting at the UIV and its adjacent proximal segment can provide insight to the anticipated biomechanical performance of a spinal fusion and spinal rod implantation. For example, failure to appropriately account for and unload these forces can lead to complications such as stress shielding, Proximal Junctional Kyphosis (PJK), Proximal Junctional Failure (PJF), and Adjacent Segment Disease of the lumbar spine or other region, resulting from excessive stress concentration and loading at the transition zone between the rigidly stabilized spine and the mobile, non-instrumented segments. Therefore, optimizing the shape, flexibility, and implantation location of the spinal rod may be useful in achieving a gradual transition of mechanical stress and reduce the risk of complications at the UIV proximal segment and to minimize, prevent, or treat complications such as stress shielding, Proximal Junctional Kyphosis (PJK), Proximal Junctional Failure (PJF), and Adjacent Segment Disease of the lumbar spine or other region, resulting from excessive stress concentration and loading at the transition zone between the rigidly stabilized spine and the mobile, non-instrumented segments.

To account for these forces, the disclosed spinal rods, systems, and methods may use computational modeling, intraoperative assessment, and in vitro biomechanical testing to analyze stress distribution and load transfer at the UIV and proximal junction. For example, the disclosed spinal rods, systems, and methods may comprise pre-planning of the pelvic incidence, sagittal balance, osteotomy angles, screw planning etc., followed by intraoperative imaging and artificial intelligence predictors to allow for corrections or changes from the pre-surgery plan to the intraoperative surgery plan leading to the end result. The disclosed spinal rods, systems, and methods may further comprise having multiple spinal rod choices to accommodate for the corrections or changes, which may lead to a better post surgical outcome. The disclosed spinal rods, systems, and methods may further comprise one or more linear segments that can be placed anywhere to address correction and bone quality. The smooth, continuously curved profile of a Bezier-defined taper eliminates abrupt changes in cross-sectional area, which would otherwise create stress risers (e.g., the "hot spots" already referenced). The tangent continuity of the Bezier curve at the junctions between the tapered segment and the adjacent linear segments ensures a gradual stiffness transition.

These methodologies can enable the assessment of various rod materials, diameters, tapering techniques, and contouring strategies to minimize peak stress concentrations. For example, the disclosed spinal rods, systems, and methods may comprise using DEXA scans, MRIs, X-rays, CT scans, or other imaging systems, Hounsfield units, and the like. Further, based on the imaging, the disclosed spinal rods, systems, and methods may consider and adapt how the spinal rod is contoured reducing metal hardening and may use adapted spinal rods comprising tapered segments (e.g., Bezier rod technology) with the linear segments and tapered segments (e.g., Bezier zones). Further, based on the imaging, the disclosed spinal rods, systems, and methods may consider and adapt the length, positioning, location, and quantity of spinal rod segments relative the spine (in addition to adaptations involving diameter, tapering, curvature, etc. as described herein). The disclosed spinal rods, systems, and methods may allow for the forces to be spread more evenly across the rods surface reducing hot spots while still allowing the rod to bend in a more natural fashion anatomically transferring forces evenly throughout the construct and reducing the forces on the neck of the screw thereby reducing screw neck breakage and reducing contact pressures, stress shielding, and micromotion. The disclosed spinal rods, systems, and methods may create an improved biomechanical construct, etc., and allow for the spinal rods to be matched to the bone, providing a tailored-to-the-patient solution and a patient-specific implants. In an embodiment, these methodologies can be carried out based on a patient’s anticipated anatomy based on preoperative planning.

For example, preoperative planning can incorporate patient-specific factors such as spinal alignment and deformity, bone mineral density, muscle imbalances, and the like to determine an optimal construct that mitigates excessive strain at the UIV. For example, patient-specific factors can include matching the spinal rod to the alignment and correction of the spine via appropriate linear segment diameters matched to the bone quality and musculature of the patient to create a patient matched, patient specific spinal rod. In an embodiment, these methodologies can be carried out based on a patient’s actual anatomy based on intraoperative assessment. For example, intraoperative fluoroscopy and navigation systems may allow for real-time assessment of spinal alignment and spinal rod placement.

In an embodiment, the disclosed spinal rods, systems, and methods may include one or more (or all) of: graduated stiffness, where the proximal segment exhibits reduced rigidity to allow for a more gradual load transition; tapered rods or multi-material constructs, incorporating more flexible materials near the UIV and stiffer materials distally, or vice versa; implantation location which follows the natural sagittal curvature of a patient; and the like. The tapered segments connecting different-material portions of the rod may also be Bezier tapered segments, and that the Bezier curve parameters (control points, curve order) may be selected to account for the differing material properties of adjacent segments and to optimize the stiffness transition between them.

In another example, the disclosed spinal rods, systems, and methods can be used to account for and match bilateral forces, which can, in turn, assist in maintaining spinal alignment, preventing asymmetric loading, and overall reducing the risk of mechanical complications. For example, forces can be assessed via preoperative and intraoperative planning by analyzing the bone quality, deformity, curvature, corrections, etc., and comparing each to either side to create custom spinal rods that accommodate to each side of the patient based on the specific anatomical needs of that patient rather than a (set of rods) that have identical bends and would not match inconsistencies bilaterally. Without such accounting, the bilateral forces can cause differential stress distribution across the vertebral column, resulting in unintended coronal or sagittal malalignment, increased implant strain, and potential rod failure. Therefore, optimizing the shape, flexibility, and implantation location of the spinal rod may be useful in achieving a balance of bilateral forces and reduce the risk of complications due to stress shielding, and the like.

To account for these forces, the disclosed spinal rods, systems, and methods may use computational modeling, intraoperative assessment, and in vitro biomechanical testing to analyze stress distribution and load transfer on each side of the spinal column. For example, the disclosed spinal rods, systems, and methods may comprise using AI, navigation, intraoperative CT, and the like. These methodologies can enable the assessment of various rod materials, diameters, tapering techniques, and contouring strategies to achieve a more even load sharing. In an embodiment, these methodologies can be carried out based on a patient’s anticipated anatomy based on preoperative planning. For example, the more severe the curve of a patient’s anatomy, the more force and manipulation of the spine may be required to correct the curve creating more stiffness and therefore increasing the overall construct stiffness and greater forces at the UIV and proximal screws. The disclosed spinal rods, systems, and methods may comprise variable spinal rod segments, lengths, diameters, changes in diameters, tapering (e.g., Bezier zones), and locations or positioning thereof, which may further allow for variable spinal rod segments, lengths, diameters, changes in diameters, tapering (e.g., Bezier zones), and locations or positioning thereof, to be placed in strategic locations to off set these forces. For example, linear segments or curved segments may be placed anywhere to achieve desired correction of the spine. The Bezier taper parameters may differ between the left-side and right-side rods to account for asymmetric anatomical conditions, and that the computational system may independently optimize the Bezier control points for each rod.

For example, preoperative planning can incorporate patient-specific factors such as spinal alignment and deformity, bone mineral density, muscle imbalances, and the like, which may contribute to asymmetric forces and necessitate strategic modifications in rod design and placement. For example, patient-specific factors can include osteotomy angles, sagittal balance, pelvic incidence, bone quality, interbody cage angles and materials, and the like. In an embodiment, these methodologies can be carried out based on a patient’s actual anatomy based on intraoperative assessment. For example, intraoperative fluoroscopy and navigation systems may allow for real-time assessment of spinal alignment and spinal rod placement.

In an embodiment, the disclosed spinal rods, systems, and methods may include one or more (or all) of: symmetric contouring that aligns with the patient’s spinal curvature; matched flexibility on each side of the spinal column; minimal force discrepancies between the left and right constructs; consistent load transmission; and reduced asymmetric strain. In an embodiment, the disclosed spinal rods, systems, and methods may include one or more (or all) of: load sharing across the construct, protecting the junctions of the thoracolumbar and cervical thoracic spine junction reducing the unloading at the UIV, while allowing for the prediction of the unloading forces to predict a failure state at the unsupported spine, and the like.

In an embodiment, the disclosed spinal rods, systems, and methods may further account for the biomechanical interrelationship between one or more interbody cages and the spinal rod to achieve a coordinated spinal construct that addresses correction across the anterior, central, and posterior columns of the spine. An interbody cage, as used herein, refers to an implantable device positioned within the intervertebral disc space to restore disc height, provide structural support, and facilitate fusion between adjacent vertebral bodies. The interbody cage may be placed via an anterior, lateral, transforaminal, or posterior approach, and may be positioned at one or more spinal levels within the construct. Because the interbody cage and the spinal rod each independently influence the biomechanical behavior of the spinal construct, matching the parameters of the interbody cage to the configuration and stiffness characteristics of the spinal rod may provide improved alignment correction, more favorable load distribution, and reduced risk of complications such as stress shielding, Proximal Junctional Kyphosis (PJK), Proximal Junctional Failure (PJF), and Adjacent Segment Disease.

In an embodiment, the interbody cage may be characterized by one or more cage parameters including, without limitation, lordotic angle, cage height, cage width, cage length, cage footprint geometry, cage curvature (e.g., sagittal and/or coronal curvature), cage material, cage modulus of elasticity, cage porosity, cage surface characteristics, endplate contact area, and angular correction capacity in the sagittal and coronal planes. For example, the lordotic angle of the interbody cage may be selected to restore or enhance segmental lordosis at the level of insertion, contributing to overall sagittal alignment correction. The cage height may be selected to restore intervertebral disc height and decompress neural elements, while also influencing the degree of segmental correction achieved. The cage modulus of elasticity may be selected based on the bone mineral density and endplate strength of the adjacent vertebral bodies to reduce the risk of subsidence while maintaining adequate load transfer through the anterior and central columns.

In an embodiment, the spinal rod may be characterized by one or more rod parameters as described herein, including, without limitation, the number, lengths, diameters, and order of the plurality of linear segments; the number, lengths, extent, and location of the at least one tapered segment (e.g., Bezier zones); the curvature of the spinal rod; the material or materials of the spinal rod; and the overall stiffness profile along the longitudinal axis of the spinal rod. Additionally, the rod parameters may include angular parameters derived from or responsive to the patient's spinal alignment, including, without limitation, pelvic incidence, sacral slope, pelvic tilt, sagittal vertical axis, sagittal balance, T1 pelvic angle (TPA), lumbar lordosis, thoracic kyphosis, segmental angles such as L4-L5 segmental lordosis, and the like.

In an embodiment, the disclosed spinal rods, systems, and methods may match the one or more cage parameters of the interbody cage to the one or more rod parameters of the spinal rod to achieve a biomechanically coordinated construct. For example, the lordotic angle and height of the interbody cage may influence the segmental correction achieved at the anterior and central columns, which in turn affects the forces and moments acting on the posterior column where the spinal rod and pedicle screws are positioned. By accounting for the cage's contribution to segmental correction, the rod parameters, including linear segment diameters, tapering, curvature, and material selection, may be determined or adjusted to complement the cage's correction and achieve a balanced load distribution across all three columns. In this manner, the anterior column correction provided by the interbody cage and the posterior column stabilization provided by the spinal rod may be coordinated to achieve an overall construct that maintains alignment while distributing forces more evenly and reducing the risk of mechanical complications at any single column or level.

In an embodiment, matching the interbody cage to the spinal rod may further account for the modulus of elasticity of the cage material relative to the stiffness profile of the spinal rod. For example, an interbody cage having a higher modulus of elasticity may provide greater anterior column support and load bearing, which may allow the spinal rod to be configured with reduced stiffness (e.g., smaller diameter linear segments, more gradual tapering, or a more flexible material) at the corresponding spinal level, thereby reducing the overall construct stiffness and the forces at the UIV and proximal screws. Conversely, an interbody cage having a lower modulus of elasticity or greater porosity may necessitate a stiffer rod configuration at that level to maintain adequate posterior column support and prevent excessive micromotion at the fusion site. The disclosed spinal rods, systems, and methods may therefore account for the interplay between cage stiffness and rod stiffness at each instrumented level to optimize the overall construct biomechanics.

In an embodiment, the cage parameters and the rod parameters may be collectively provided as inputs into the system 1000, or a portion thereof, as described herein. For example, the cage parameters, including lordotic angle, cage height, cage modulus of elasticity, cage footprint geometry, endplate contact area, and angular correction capacity, may be entered into the system 1000 as additional inputs alongside the preoperative and intraoperative assessment data described herein (e.g., imaging data such as MRI, CT, and DEXA scans; physician observations; diagnoses; anatomical dimensions of the patient; osteotomy angles; sagittal balance; pelvic incidence; and the like). The system 1000, utilizing the processor 500 and the algorithm or criteria or standards described herein, may analyze the cage parameters in conjunction with the patient-specific anatomical and alignment data to determine a spinal rod configuration that is biomechanically matched to the selected interbody cage or cages.

In an embodiment, the system 1000 may determine the spinal rod configuration by calculating or estimating the contribution of the interbody cage to segmental correction (e.g., the amount of lordosis restoration, disc height restoration, and coronal correction provided by the cage at each level) and then determining the rod parameters needed to complement that correction at the posterior column. For example, if a lordotic interbody cage is placed at L4-L5 and provides a specified number of degrees of segmental lordosis restoration, the system 1000 may determine the appropriate rod curvature, segment diameters, tapering, and material at the L4-L5 level to accommodate the corrected alignment and to ensure that the forces at the posterior column are balanced against the anterior column correction provided by the cage. The system 1000 may similarly account for the cage's effect on global spinal alignment parameters, such as the impact of the cage's lordotic angle on overall lumbar lordosis, sagittal vertical axis, pelvic tilt, and T1 pelvic angle (TPA), and adjust the rod configuration accordingly to achieve the desired postoperative alignment targets.

In an embodiment, the system 1000 may generate an integrated construct output that includes both the recommended interbody cage parameters and the matched spinal rod configuration. For example, the integrated construct output may include a curvature template for the spinal rod, as described herein, that accounts for the presence and parameters of the interbody cage at one or more levels, along with a failure-state risk percentage that reflects the combined biomechanical performance of the cage and rod construct. The failure-state risk percentage may account for, without limitation, the risk of cage subsidence, rod fracture, screw loosening, PJK, PJF, adjacent segment disease, pseudarthrosis, and loss of correction, based on the interplay between the cage parameters, the rod parameters, the patient's bone quality, and the patient's spinal alignment.

In an embodiment, the system 1000 may further allow for manipulation of the cage parameters, the rod parameters, or both, to evaluate the effect of different construct configurations on the predicted outcomes and failure-state risk. For example, a surgeon may adjust the lordotic angle, height, or modulus of the interbody cage within the system 1000 and observe the corresponding changes to the recommended rod configuration and the failure-state risk percentage, or vice versa. This iterative manipulation of parameters may allow the surgeon to evaluate multiple construct configurations, including different combinations of cage size, cage material, rod segment diameters, rod tapering, rod curvature, and rod material, to identify an optimal or preferred surgical plan that achieves the desired alignment correction across all three spinal columns while minimizing the predicted risk of mechanical complications and failure states.

In an embodiment, the matching of the interbody cage to the spinal rod may also account for the bilateral forces described herein. For example, an interbody cage placed asymmetrically (e.g., via a transforaminal approach from one side) may introduce asymmetric loading across the anterior column, which may necessitate different rod configurations on each side of the spinal column to maintain symmetric load transmission across the posterior column. The system 1000 may account for this asymmetry by determining a first spinal rod configuration for a first side of the patient's spinal column and a second spinal rod configuration for a second side of the patient's spinal column, each matched to the cage parameters and the resulting bilateral force distribution, to provide symmetric or balanced load transmission across the construct.

In an embodiment, the matching of the interbody cage to the spinal rod may further account for multi-level constructs in which interbody cages are placed at two or more spinal levels. In such embodiments, the system 1000 may analyze the cumulative effect of the interbody cages at the multiple levels on the overall spinal alignment and force distribution and determine a spinal rod configuration that accounts for the aggregate correction provided by all cages within the construct. For example, if a first interbody cage is placed at L4-L5 and a second interbody cage is placed at L5-S1, the system 1000 may determine the rod segment lengths, diameters, tapering, and curvature to complement the segmental correction at each level while maintaining the desired overall sagittal balance, pelvic incidence-lumbar lordosis mismatch target, and T1 pelvic angle.

In an embodiment, the output instructions generated by the system 1000 for the matched interbody cage and spinal rod construct may include, without limitation, one or more of: recommended cage type, cage size, cage lordotic angle, cage material, and cage placement approach for each instrumented level; recommended rod segment lengths, diameters, tapering (e.g., Bezier zones), curvature, and material; recommended rod contouring, rod rotation, sequential compression and distraction, rod translation, rod stiffness modulation, cross-link placement, screw type and placement selection, and rod cutting and end modification as described herein; and a predicted failure-state risk percentage and failure mode analysis for the integrated construct. The output instructions may further include alternative construct configurations that the surgeon may select from, each with corresponding predicted outcomes and failure-state risk assessments, allowing the surgeon to make an informed decision on the optimal surgical plan for the patient.

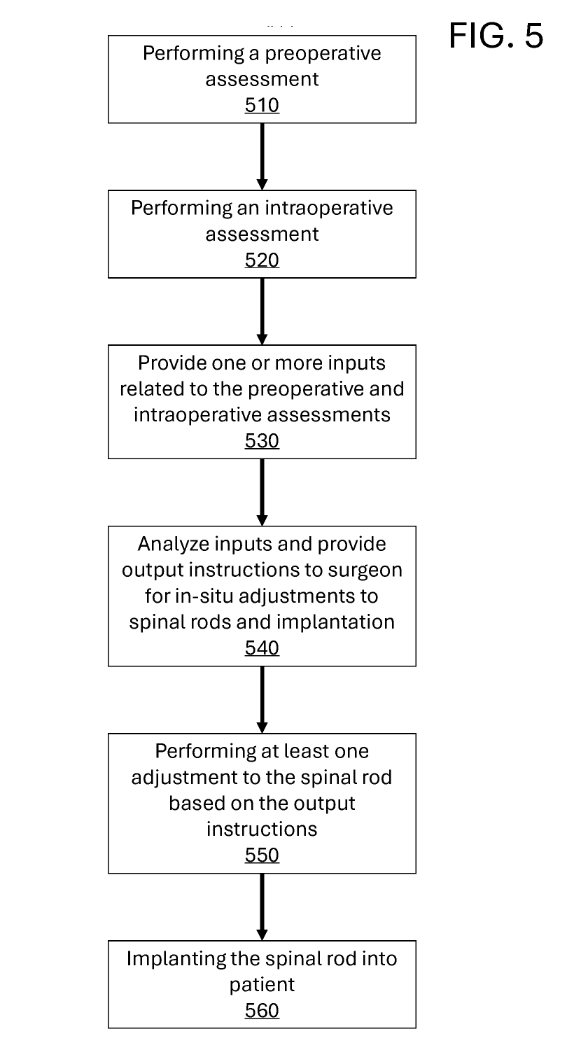

As described herein and shown in FIG. 5, the disclosed spinal rods, systems, and methods may use individually or a combination of preoperative assessment and in-situ or intraoperative adjustment to provide a patient specific spinal construct adapted to the patient’s morphology. Moreover, the disclosed spinal rods, systems, and methods may facilitate in-situ adjustments to get to neutral and address current anatomy as well as to prevent future disease states. For example, preoperative assessments may include imaging such as CT or MRI scans, and the like, to assess and evaluate the disease state and to determine what needs to be corrected what procedures can be performed to bring the spine back to the neutral position. As described herein, the spinal rods may have certain segment attributes, such as variable length, fixed diameter, tapering (e.g., Bezier zones), curvature, etc., to bring the spine back to the neutral position while accounting for certain forces, etc., to provide an improved long-term outcome. For example, intraoperative assessments may include imaging during the procedure, such as CT scans, and the like, that will accommodate for rotation and the corrections made already made and allow for assessment of the preoperative plan against the current status. As described herein, the spinal rods can then be adapted or modified to match a spinal rods to the intraoperative assessment if needed.

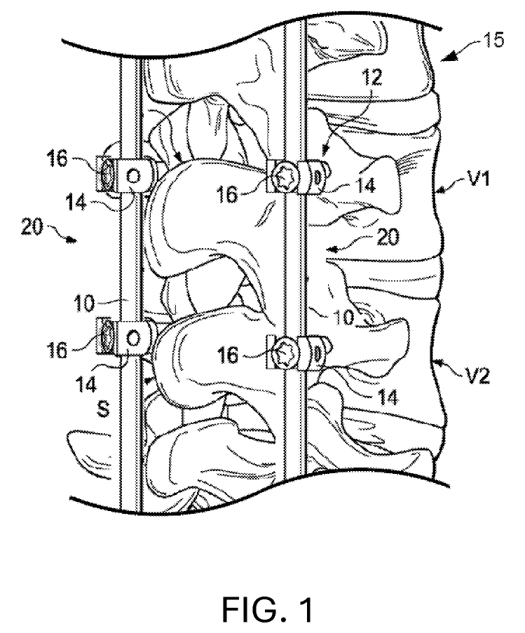

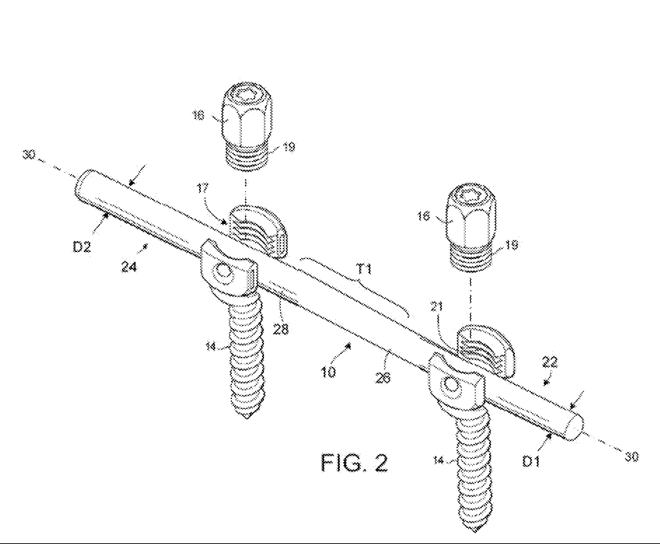

With reference to FIG. 1, each first and second stabilization systems 20 includes a spinal rod 10 that has a plurality of linear regions D1, D2 (as illustrated in FIG. 2) with a different cross-sectional dimension attached to vertebral members V1, V2 by pedicle assemblies 12. Each pedicle assembly 12 includes a pedicle screw 14 and a retaining cap 16. To couple the spinal rod 10 to the vertebral members V1, V2, the pedicle screw 14 is threaded into an aperture formed in one of the vertebral members V1, V2. A saddle 17 (as illustrated in FIG. 2) of each pedicle assembly 12 receives a portion of the spinal rod 10. The saddle 17 can be formed as a generally U-shaped recess at a proximate end of the pedicle screw 14 that remains exposed externally of the bone while the pedicle screw 14 is installed in one of the vertebral members V1, V2. Threading 19 provided to a proximate end of the retaining cap 16 cooperates with threading 21 formed along an interior surface of the saddle 17 to exert a compressive force on the spinal rod 10, thereby securing the spinal rod 10 in the saddle 17.

The spinal rods 10 in FIG. 1 are positioned at a posterior side of the spine 15, on opposite sides of the spinous processes S. Spinal rods 10 may be attached to the spine 15 at any other location to be stabilized, such as lateral and anterior locations for example. The spinal rods 10 may also be attached at various sections of the spine, including the base of the skull and to vertebrae in the cervical, thoracic, lumbar, and sacral regions. Thus, the illustration in FIG. 1 is provided merely as a representative example of one application of a spinal rod 10.

In the exemplary assembly 20, the spinal rods 10 are secured to vertebral members V1, V2 by pedicle assemblies 12 comprising a pedicle screw 14 and a retaining cap 16. The outer surface of the spinal rod 10 is grasped, clamped, or otherwise secured between the pedicle screw 14 and retaining cap 16. In some embodiments, these are multi-axial pedicle screws. Other mechanisms for securing spinal rods 10 to vertebral members V1, V2 include hooks, cables, and other such devices. Further, examples of other types of retaining hardware include threaded caps, screws, and pins. The spinal rods 10 are also attached to plates in other configurations. In some examples, interbody devices or implants, fusion or dynamic, may be disposed between the adjacent vertebrae. Thus, the exemplary assemblies 20 shown in FIG. 1 are merely representative of one type of attachment mechanism.

The configuration of the stabilization system 20, in which the spinal rod 10 having different rod regions 22, 24 with different diameters D1, D2 is secured to vertebral members V1, V2 by commonly-sized pedicle assemblies 12, provides a variable stiffness along the length of the spinal rod 10 that is tailored to the respective vertebral members being stabilized. For example, rod regions having a larger diameter may provide greater rigidity at vertebral levels requiring increased stabilization, while rod regions having a smaller diameter may provide reduced rigidity at vertebral levels where a more gradual load transition is desired, such as near the Upper Instrumented Vertebra (UIV). The tapered transition region T1 disposed between adjacent rod regions 22, 24 further distributes force across the surface of the spinal rod 10 at the transition between regions of different stiffness, reducing stress concentrations and "hot spots" that could otherwise occur at an abrupt change in diameter. In this manner, the variable stiffness provided by the different rod regions 22, 24 and the tapered transition region T1 translates to different force profiles at each pedicle screw 14 location along the spinal rod 10, allowing the forces at each anchor point to be managed according to the bone quality, anatomical dimensions, and alignment characteristics of the patient at that vertebral level. Further, because the commonly-sized pedicle assemblies 12, including a common saddle 17 size and common retaining cap 16 size, accommodate the different rod diameters D1, D2, the spinal rod 10 may be configured with any combination of segment diameters, tapering, and lengths without requiring specialized or diameter-specific pedicle hardware, thereby supporting the configurability of the spinal rod 10 to a patient's anatomy as described herein.

As shown in FIG. 2, the spinal rod 10 includes a plurality of linearly-arranged rod regions 22, 24 separated by a tapered transition region T1 (e.g., Bezier zones). The rod regions 22, 24 and the tapered region T1 can be integrally formed together as portions of the same monolithic structure, which can be substantially cylindrical in shape and have a generally circular or elliptical cross section. In other embodiments, the spinal rods are formed of separate portions that secured together. Embodiments of the spinal rod 10 are formed from surgical stainless steel, and can have any desired length to stabilize the region of the spine of interest. For example, the spinal rod 10 can have a length of up to 600 mm, up to 550 mm, up to 500 mm, up to 450 mm, up to 400 mm, up to 375 mm, up to 350 mm, etc. The spinal rod 10 is described herein as having a circular cross section for the sake of brevity and clarity, but other cross-sectional shapes can also be utilized to provide the spinal rod 10 with desired rigidity and other physical properties without departing from the scope of the present disclosure.

The circular cross section of the different regions 22, 24 can each have a different diameter D1, D2. The different diameters D1, D2 (or other dimension of the rod regions 22, 24 if the cross section is not circular) can be chosen to provide the respective regions with a desired rigidity to mitigate the effects of fatigue on the life of the implant due to movement of the respective spine segments. For example, the diameters D1, D2 can each be independently selected to be approximately 3.75 mm, 4.00 mm, 4.25 mm, 4.50 mm, 4.75 mm, 5.00 mm, 5.25 mm, 5.50 mm, 5.75 mm, any other diameter within a range from approximately 3.00 mm to about 6.00 mm, including any subrange therein. Examples of such subranges can be up to 1.00 mm in size (e.g., D1 is approximately 4.50 mm and D2 is approximately 5.50 mm); up to 0.75 mm in size (e.g., D1 is approximately 4.50 mm and D2 is approximately 5.25 mm); up to 0.50 mm in size (e.g., D1 is approximately 4.50 mm and D2 is approximately 5.00 mm); etc. Having the approximate diameters above allows for machining tolerances of up to ±10%.

Despite the different diameters D1, D2 of the different rod regions 22, 24, embodiments of the present stabilization system 20 can include commonly-sized pedicle assemblies 12, or portions thereof, to couple the spinal rod 10 to the vertebral members V1, V2. For example, pedicle screws 14 having a common saddle 17 size and common retaining cap 16 size can be installed along each of the rod regions 22, 24. Thus, the saddle 17 and retaining cap 16 can be sized to accommodate the largest of the diameters D1, D2, but create a range of adjustment that is suitable to allow the retaining cap 16 to secure the smallest of the diameters D1, D2 within the saddle 17.

Forming different rod regions 22, 24 of the same spinal rod 10 with different diameters D1, D2 affords the different rod regions 22, 24 with different rigidity, tailored to the respective vertebral members V1, V2 to be stabilized by those rod regions 22, 24. To at least partially mitigate stress concentrations along the spinal rod 10, a tapered region T1 forms a gradual transition between the different diameters D1, D2 of the rod regions 22, 24. Instead of a full, step change in the diameter at a point location along the length of the spinal rod 10, the tapered region T1 elongates the portion of the spinal rod 10 over which the diameter changes from the diameter D1 to the diameter D2. For example, a first end 26 of the tapered region T1 can have a diameter that is approximately the same as diameter D1, while a second end 28 of the tapered region T1 can have a diameter that is approximately the same as diameter D2. The first and second ends 26, 28 of the tapered region T1 can be separated from each other by at least 5 mm, at least 10 mm, at least 15 mm, at least 20 mm, at least 25 mm, at least 30 mm, at least 35 mm, at least 40 mm, etc. along a longitudinal axis 30 of the spinal rod 10.

Embodiments of the tapered region T1 can be limited in length along the longitudinal axis 30 to less than a defined threshold such as 50 mm, for example, to allow the individual rod regions 22, 24 to have a substantially cylindrical shape. Thus, spinal rod 10 can be formed from a plurality of cylindrical rod regions 22, 24, and a tapered transition region, optionally giving the spinal rod 10 a stepped shape instead of a conical shape, with continuous angled sides along the entire length of the spinal rod 10.

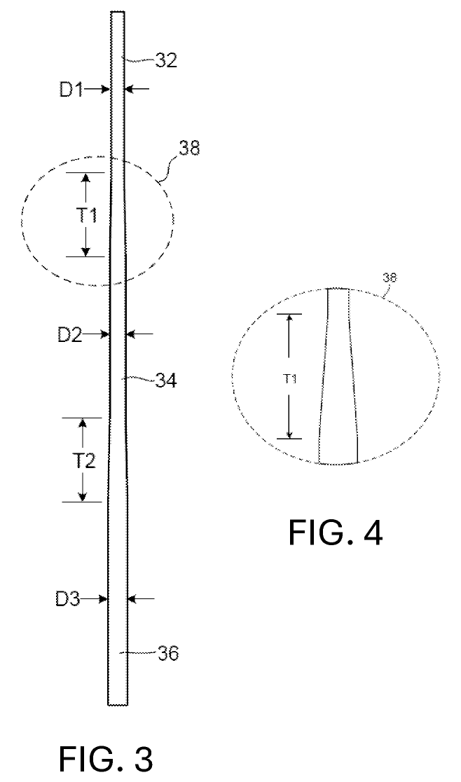

The embodiment of the spinal rod 10 in FIG. 2 has two rod regions 22, 24 having different diameters D1, D2 separated by a tapered region T1, however the present disclosure is not so limited. FIG. 3 shows another embodiment of the spinal rod 10 that includes three rod regions 32, 34, 36. Similar to the above embodiments, each rod region 32, 34, 36 can be cylindrical in shape with a substantially constant diameter D1, D2, D3 such as those described above. Tapered regions T1, T2 separate the rod regions 32, 34, 36 from each other. The spinal rod 10 can be formed integrally with the regions or the regions can be formed separately and secured together.

Because the difference in diameter between rod regions 32, 34, 36 is subtle, and may be difficult to discern from the drawings, FIG. 4 shows an enlarged view of the tapered region T1 between the diameters D1, D2 enclosed by broken lines 38 in FIG. 3. The rate at which the diameter changes between diameters D1, D2 can optionally be constant across the length of the tapered region T1. Thus, as shown in FIG. 4, the tapered region T1 forms a substantially frusto-conical shape between the rod regions 32, 34. The tapered region T1 affords surgeons the flexibility to install the pedicle assemblies 12 at any location along the length of the spinal rod 10, including along the tapered region T1 or at an intersection of the tapered region T1 and one of the rod regions 32, 34.

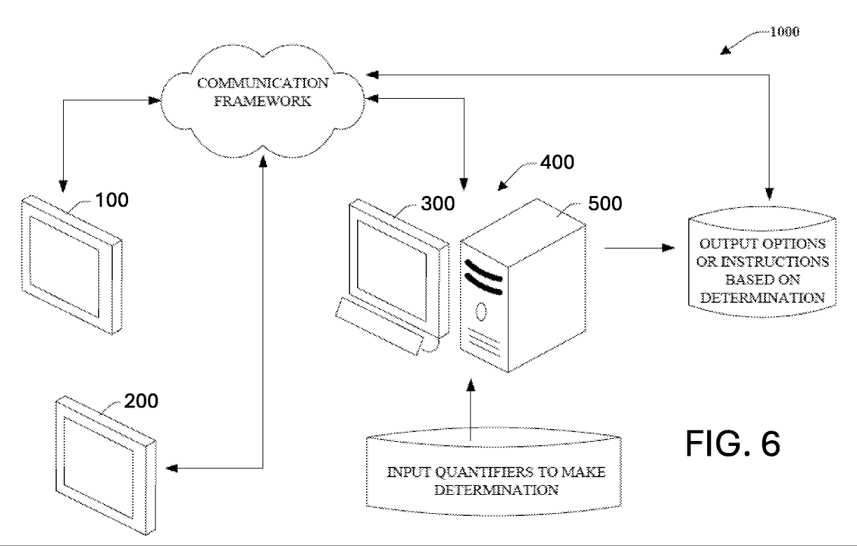

Having described the structural features of the spinal rod 10 and the various configurations of linear regions, tapered regions, and cross-sectional dimensions thereof with reference to FIGS. 1–4, the following description now turns to the systems and methods by which the spinal rod configuration may be determined for a particular patient and subsequently implanted. As described above, the lengths, diameters, tapering, location, number of segments, curvature, and combinations thereof may be adapted to a patient's anatomy to achieve an optimal biomechanical construct. FIGS. 5 and 6 illustrate an exemplary method and system, respectively, for receiving patient-specific data, determining an appropriate spinal rod configuration based on that data, and providing output instructions for in-situ adjustment or implantation of the spinal rod.

Referring now to FIG. 5, the disclosed spinal rods, systems, and methods may include a method for determining curvature and placement of a spinal rod in a patient. The method may include the step 510 of performing a preoperative assessment on the patient, the step 520 of performing an intraoperative assessment on the patient, the step 530 of providing one or more inputs related to the preoperative assessment and the intraoperative assessment into a system, the step 540 of analyzing the inputs and providing an output, the step 550 of performing at least one adjustment to the spinal rod based on the output, and the step 560 of implanting the spinal rod into the patient. Each of these steps is described in further detail below.

At step 510, a preoperative assessment may be performed on the patient. The preoperative assessment may include obtaining imaging data of the patient's spine, such as MRI, CT, DEXA scans, X-rays, and the like, to assess and evaluate the disease state and to determine what corrections are needed to bring the spine back to a neutral position. The preoperative assessment may further include evaluating patient-specific factors such as spinal alignment and deformity, bone mineral density, muscle imbalances, osteotomy angles, sagittal balance, pelvic incidence, bone quality, interbody cage angles and materials, and the like. Based on these evaluations, a preoperative plan may be developed that identifies the type, configuration, and placement of the spinal rod and associated hardware, including the number, lengths, diameters, and order of the plurality of linear segments, the number, lengths, extent, and location of the at least one tapered segment (e.g., Bezier zones), the curvature of the spinal rod, and the material or materials of the spinal rod.

At step 520, an intraoperative assessment may be performed on the patient. The intraoperative assessment may include imaging during the procedure, such as CT scans, intraoperative fluoroscopy, navigation systems, and the like, that will accommodate for rotation and the corrections already made and allow for assessment of the preoperative plan against the current status of the patient's anatomy. In an embodiment, the spinal rod configuration may be adapted or modified based on the intraoperative assessment if the patient's intraoperative anatomy differs from the preoperative plan.

At step 530, one or more inputs related to the preoperative assessment and the intraoperative assessment may be provided into a system, such as system 1000 described herein with reference to FIG. 6, or a portion thereof. The one or more inputs may include, without limitation, imaging data such as MRI, CT, DEXA scans, and X-rays; physician or surgeon observations or notes; diagnoses; dimensions or other characteristics of the patient's anatomy; tolerances; osteotomy angles; sagittal balance parameters such as pelvic incidence, sacral slope, pelvic tilt, sagittal vertical axis, and T1 pelvic angle; interbody cage parameters such as lordotic angle, cage height, cage modulus of elasticity, and cage material; and the like. The inputs may be provided at different times, by different users, and through different devices within the system 1000.

At step 540, the system may analyze the one or more inputs and provide an output such as instructions to a surgeon for in-situ adjustments to the spinal rod or implantation of the spinal rod. It is noted that step 540 may be accomplished by the system 1000, or a portion of system 1000 thereof, utilizing the processor 500 and an algorithm or certain criteria or standards as described herein. Alternatively, step 540 may be accomplished by the surgeon manually by following criteria or standards for determining possible in-situ adjustments to the spinal rod or implantation of the spinal rod based on patient anatomy and desired results as described herein. In an embodiment, the analysis at step 540 may include determining stress distribution, load transfer, and other biomechanical factors associated with the patient's spine, and may utilize one or more of computational modeling, artificial intelligence, intraoperative navigation, or intraoperative CT. The output may include a spinal rod configuration comprising a plurality of linear segments and at least one tapered segment configured to provide variable stiffness, as well as a curvature template based on the spinal rod configuration. In an embodiment, the curvature template may comprise a failure-state risk percentage that translates the predicted unloading forces at the UIV into a risk of a failure state, allowing the surgeon to make an informed decision on further surgical pathways.

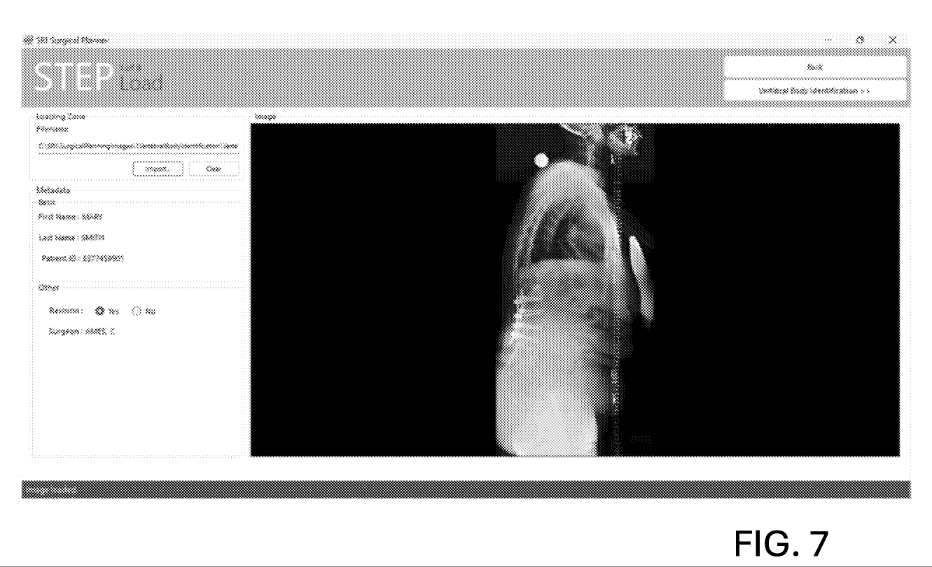

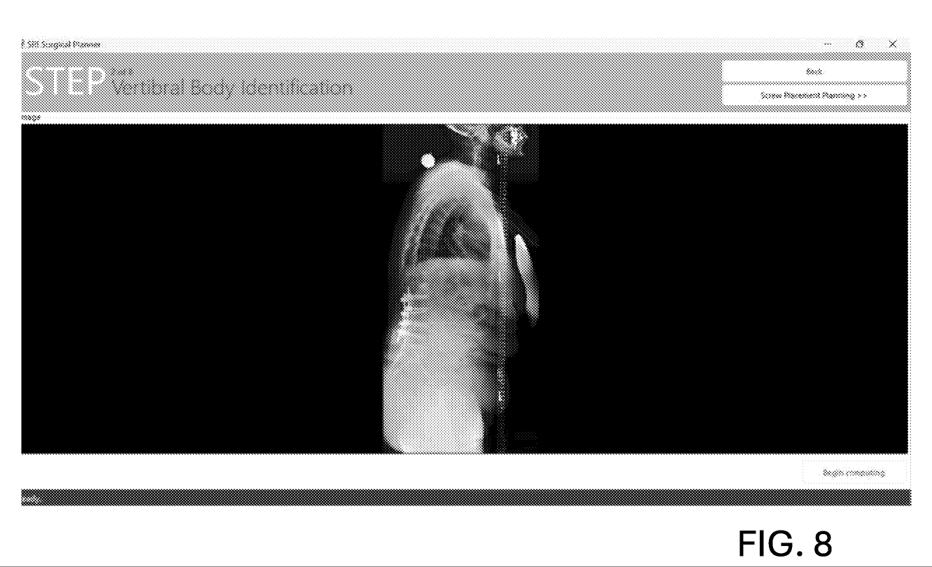

At step 550, based on the output instructions from step 540, at least one adjustment may be performed on the spinal rod. The at least one adjustment may include one or more of rod contouring (e.g., bending or shaping of the spinal rod), rod rotation, sequential compression and distraction, rod translation, rod stiffness modulation (e.g., selecting different materials having different flexibility and rigidity for all or a particular portion of the spinal rod), cross-link placement, screw type and placement selection, or rod cutting and end modification, as described in further detail herein. At step 560, the spinal rod may be implanted in the patient. In an embodiment, the method may further include implanting a second spinal rod on an opposite side of the patient's spinal column, wherein the spinal rod and the second spinal rod are configured to provide symmetric load transmission across the spinal column, as described herein with respect to bilateral force matching. In an embodiment, the described method may further include one or more of importing an image, identifying the vertebral body, screw placement planning, pelvic tilt correction, correction for sagittal imbalance, osteotomy angles, simulation of the osteotomy angles, and matching the best fit custom spinal rod to the patient. See, for example, FIGS. 7–15.

Shown in FIGS. 7–15 are exemplary steps and images that may be used or provided as a component of the disclosed spinal rods, systems, and methods. The steps illustrated in FIGS. 7–15 may correspond to sub-steps or components of one or more of steps 510–560 described above with reference to FIG. 5. FIGS. 7–15 may represent screens, user interfaces, or outputs displayed on screen 300 of device 400, or on another device within system 1000, during the preoperative planning or intraoperative assessment process.

Referring to FIG. 7, an image of the patient's spine may be uploaded or imported into the system 1000. The image may comprise one or more of an X-ray, MRI, CT, or DEXA scan, or the like, and may depict the patient's spinal anatomy in one or more views (e.g., lateral, anteroposterior, or sagittal). The imported image may serve as the baseline anatomical reference upon which the subsequent steps of vertebral identification, screw planning, alignment correction, and rod selection are performed.

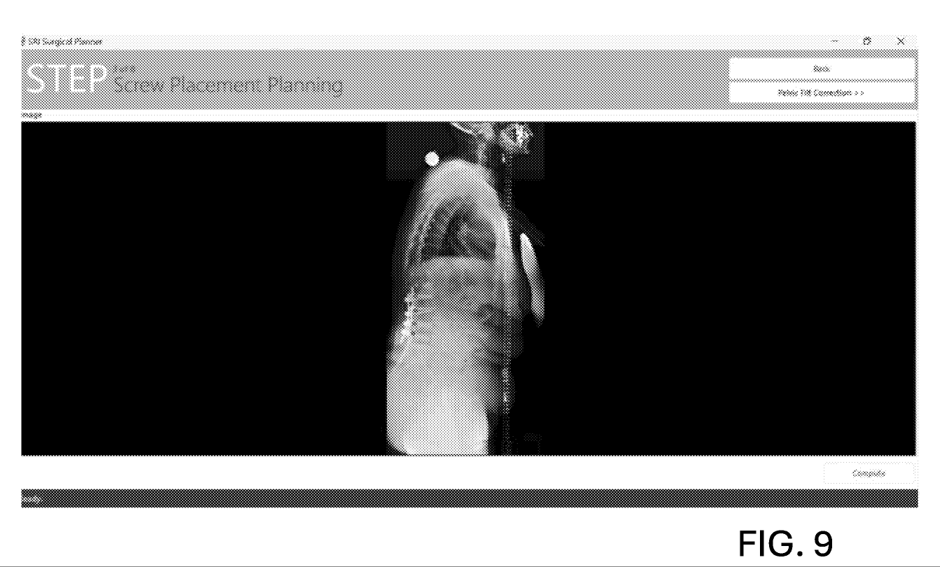

Referring to FIG. 8, the system 1000 may perform vertebral body identification on the imported image. For example, the system 1000 may automatically identify and label each vertebral body within the image, or the surgeon may manually identify and label each vertebral body, or a combination thereof. Vertebral body identification may allow the system 1000 to establish reference points along the spinal column that are used in subsequent steps for screw placement planning, alignment measurements, and rod configuration determination. In an embodiment, the system 1000 may utilize artificial intelligence or pattern recognition algorithms to assist in vertebral body identification.

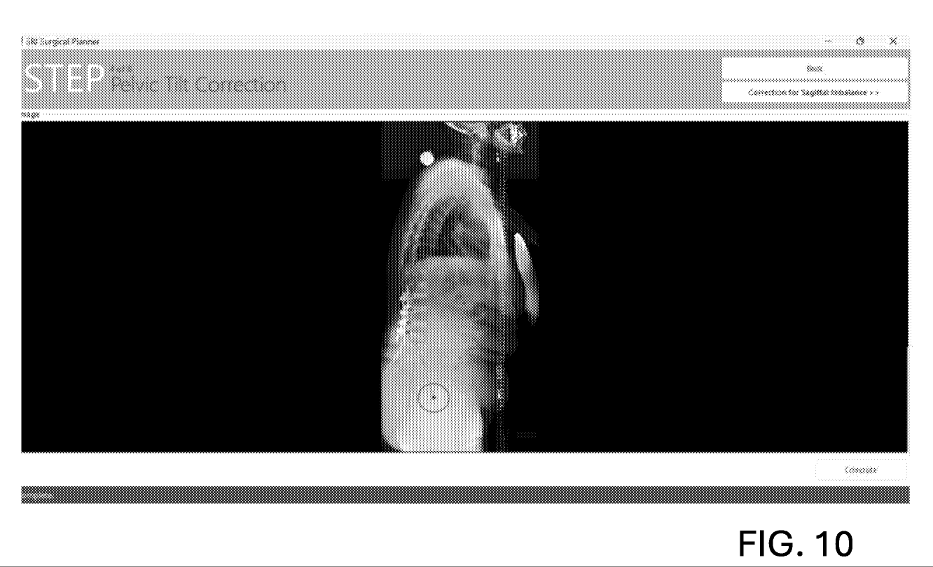

Referring to FIG. 9, the system 1000 may perform or facilitate screw placement planning. For example, the system 1000 may display recommended screw locations along the identified vertebral bodies based on the patient's anatomy, bone quality, and intended correction. The surgeon may adjust, add, or remove screw placement locations within the system 1000 based on clinical judgment. The screw placement planning may define the anchor points for the spinal rod and may influence the curvature template and rod configuration determined by the system 1000, as described herein. Once the screw placement planning has been made, the locations can allow a surgeon to visualize the curvature of the potential spinal rod to be placed.

Referring to FIG. 10, the system 1000 may perform or facilitate pelvic tilt correction. For example, the system 1000 may calculate the patient's pelvic tilt based on the imported imaging data and patient-specific parameters such as pelvic incidence and sacral slope, and may display a recommended pelvic tilt correction value. The pelvic tilt correction may be used to adjust the target spinal alignment parameters that drive the rod configuration determination in subsequent steps.

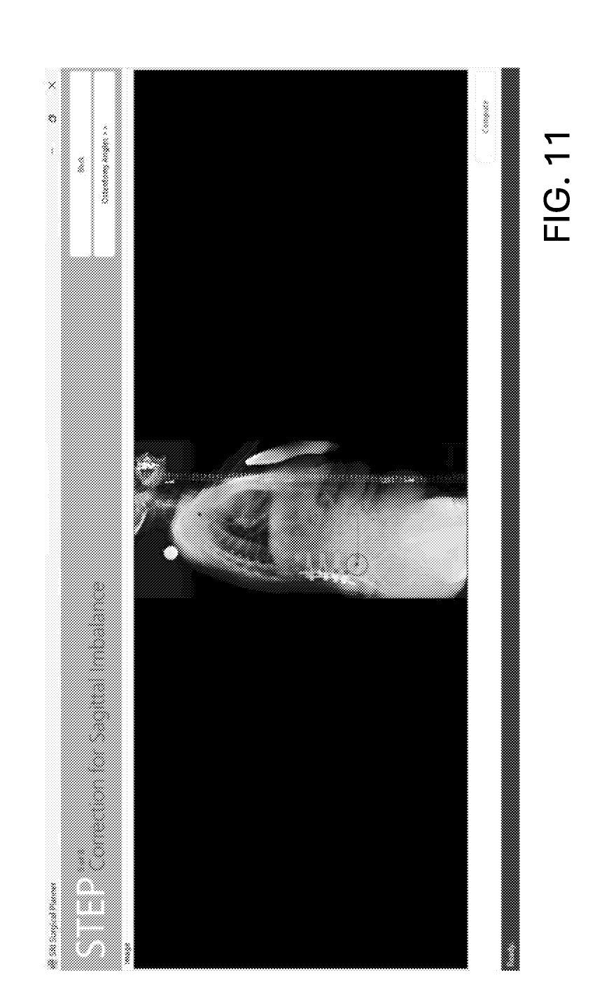

Referring to FIG. 11, the system 1000 may perform or facilitate correction for sagittal imbalance. For example, the system 1000 may calculate the patient's sagittal vertical axis (SVA), T1 pelvic angle (TPA), and pelvic incidence-lumbar lordosis (PI-LL) mismatch based on the imaging data and patient-specific parameters, and may display recommended correction targets. The sagittal imbalance correction may inform the overall curvature and stiffness profile of the spinal rod needed to achieve the desired postoperative alignment.

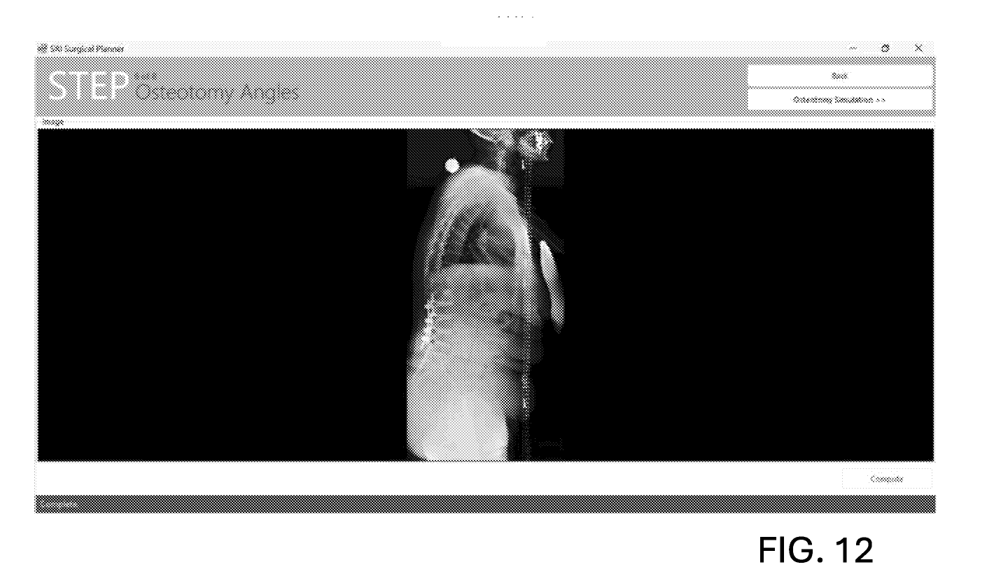

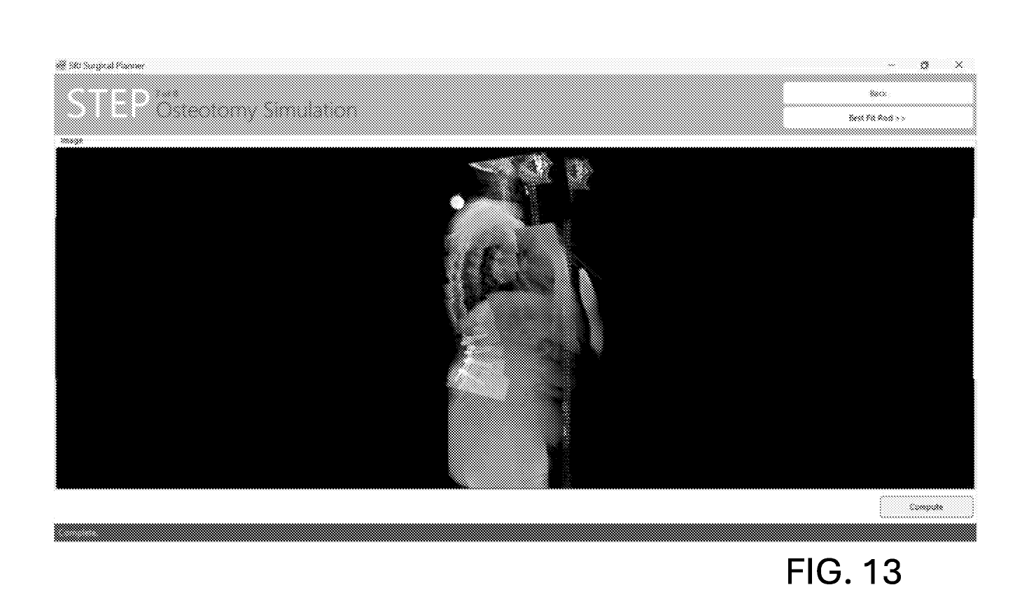

Referring to FIGS. 12 and 13, the system 1000 may perform or facilitate osteotomy angle planning and osteotomy simulation. For example, the system 1000 may allow the surgeon to input or adjust osteotomy angles at one or more vertebral levels, and may simulate the effect of the planned osteotomies on the patient's overall spinal alignment. The osteotomy simulation may allow the surgeon to visualize the anticipated postoperative alignment and to evaluate whether the planned osteotomies, in combination with the proposed rod configuration and interbody cage parameters (if applicable), achieve the desired correction targets.

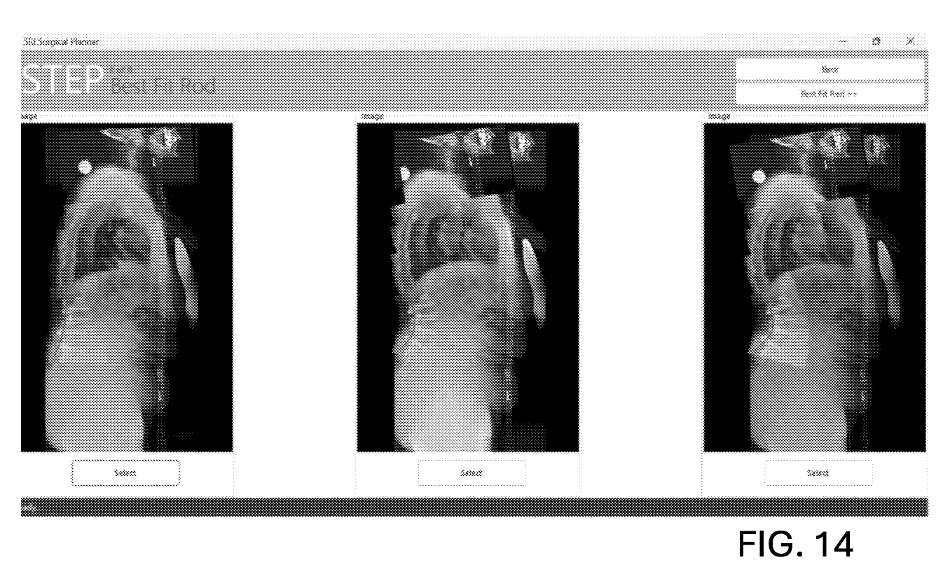

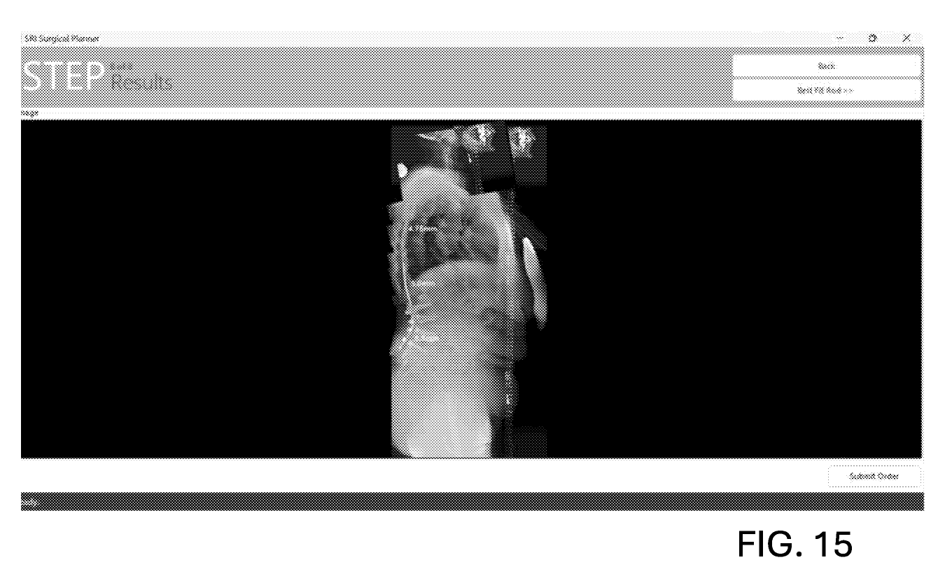

Referring to FIGS. 14 and 15, the system 1000 may output one or more options for a best fit spinal rod based on the patient's anatomy and the results of the preceding steps, including vertebral body identification, screw placement planning, pelvic tilt correction, sagittal imbalance correction, and osteotomy simulation. The output may include multiple spinal rod configuration options, each comprising recommended segment lengths, diameters, tapering (e.g., Bezier zones), curvature, and material, along with corresponding predicted outcomes and failure-state risk assessments. The surgeon may add more inputs, adjust parameters, or select an output with instructions for the type, shape, and segments of the spinal rod and the placement of the spinal rod and screws relative the patient anatomy. In an embodiment, the system 1000 may further output a curvature template for each spinal rod configuration option that allows the surgeon to compare the predicted biomechanical performance and failure-state risk of each option before making a final selection.

As shown in FIG. 6, the disclosed spinal rods, systems, and methods may utilize a system 1000, which may include a communication framework or network, one or more devices 100, 200, 400 such as a computer, phone, tablet, or the like, which may further comprise a screen 300 and a processor 500, for instance in the case of device 400, and which further may communicate to other devices within the system 1000 over the communication framework or network. In an embodiment, the system 1000 may operate as a standalone system using a single device, such as device 400 including the processor 500, without requiring connection to additional devices or a communication network. In another embodiment, the system 1000 may operate in a networked configuration in which multiple devices 100, 200, 400 communicate over the communication framework or network to share inputs, outputs, and processing tasks. For example, device 100 may be a patient-facing device configured to receive patient-provided inputs such as medical history, symptoms, or prior imaging; device 200 may be an intraoperative device configured to receive real-time imaging data, navigation data, or surgeon observations during a surgical procedure; and device 400 may be a primary processing device comprising the processor 500 and the screen 300 configured to aggregate, analyze, and display inputs and outputs to the surgeon.

The system 1000 may further comprise input and output capabilities. For example, inputs may include preoperative and intraoperative scans, photos, or the like, such as MRI, CT, DEXA scans, X-rays, and the like; physician or surgeon observations or notes; diagnoses; dimensions or other characteristics of the patient's anatomy; tolerances; osteotomy angles; sagittal balance parameters such as pelvic incidence, sacral slope, pelvic tilt, sagittal vertical axis, and T1 pelvic angle; interbody cage parameters such as lordotic angle, cage height, cage modulus of elasticity, and cage material; and the like. The system 1000 may comprise a non-transitory computer-readable medium (e.g., memory, storage) that stores instructions executed by the processor 500. The non-transitory computer-readable medium may store one or more algorithms, criteria, or standards for analyzing the received inputs and generating outputs, including spinal rod configurations, curvature templates, failure-state risk percentages, and instructions for in-situ adjustment or implantation, as described herein.

The processor 500 may execute the stored instructions to receive, aggregate, and analyze inputs provided at different times, by different users, and through different devices within the system 1000, and to generate outputs in real-time or near real-time during a surgical procedure. In an embodiment, the system 1000 may output a plurality of spinal rod configuration options based on the patient's anatomy, allowing the surgeon to evaluate and select from multiple configurations, each with corresponding predicted outcomes and failure-state risk assessments. The system 1000 may further utilize artificial intelligence, navigation, intraoperative CT, and computational modeling to analyze stress distribution, load transfer, and other biomechanical factors associated with the patient's spine, and to determine a spinal rod configuration that accounts for bilateral forces across the patient's spinal column.

The system 1000 may utilize one or more computational approaches to determine the spinal rod configuration, generate the curvature template, calculate the failure-state risk percentage, and provide output instructions as described herein. In an embodiment, the system 1000 may utilize finite element analysis (FEA) to model the biomechanical behavior of the spinal rod within the patient's spinal anatomy. For example, the system 1000 may generate a finite element model of the patient's spinal column based on the imaging data (e.g., CT, MRI), bone mineral density data (e.g., DEXA), and patient-specific anatomical dimensions received as inputs. The finite element model may represent the vertebral bodies, intervertebral discs, ligaments, pedicle screws, interbody cages (if applicable), and the spinal rod, and may simulate the mechanical interaction between these components under physiological loading conditions. The system 1000 may apply the finite element model to evaluate stress distribution, strain, contact pressures, and load transfer at each vertebral level and at each anchor point along the spinal rod, including the UIV and proximal screws. The results of the finite element analysis may be used to determine the optimal combination of linear segment lengths, diameters, tapering (e.g., Bezier zone parameters), curvature, and material for the spinal rod configuration, and to predict the unloading forces used in generating the curvature template and failure-state risk percentage.

In other embodiments, the system 1000 may additionally or alternatively utilize rule-based optimization to determine the spinal rod configuration. For example, the system 1000 may store a set of predefined rules, criteria, or standards that map patient-specific input parameters (e.g., bone mineral density ranges, spinal alignment measurements, vertebral body dimensions, deformity severity, correction targets) to corresponding rod configuration parameters (e.g., segment diameters, segment lengths, taper lengths, Bezier curve control points, material selection). The rules may be derived from clinical literature, biomechanical studies, in vitro testing data, cadaveric testing data, or surgeon experience, and may be updated over time as new data becomes available. The system 1000 may apply the stored rules to the received inputs to generate one or more candidate spinal rod configurations, and may rank the candidate configurations based on one or more optimization criteria such as minimizing predicted stress at the UIV, minimizing the failure-state risk percentage, maximizing construct stiffness within a target range, or achieving a target sagittal alignment correction.

In an embodiment, the system 1000 may additionally or alternatively utilize artificial intelligence, including machine learning, to determine the spinal rod configuration, generate the curvature template, and calculate the failure-state risk percentage. For example, the system 1000 may employ one or more trained machine learning models, such as a neural network (e.g., a deep neural network, a convolutional neural network, a recurrent neural network), a random forest, a support vector machine, a gradient boosting model, or the like, that receive as input the patient-specific data (e.g., imaging data, bone mineral density, spinal alignment parameters, anatomical dimensions, osteotomy angles, interbody cage parameters) and generate as output one or more of a recommended spinal rod configuration, a curvature template, a failure-state risk percentage, or output instructions for in-situ adjustment or implantation.

The one or more machine learning models may be trained using a training dataset comprising historical patient data. The training dataset may include, without limitation, preoperative imaging data, preoperative spinal alignment measurements, patient demographic information (e.g., age, weight, height, body mass index), bone mineral density data, diagnoses, surgical plans (e.g., screw placement locations, osteotomy angles, interbody cage parameters), spinal rod configurations used (e.g., segment lengths, diameters, tapering, materials, curvature), intraoperative measurements and observations, and postoperative outcomes (e.g., postoperative alignment measurements, fusion status, complication occurrence such as PJK, PJF, adjacent segment disease, rod fracture, screw loosening, pseudarthrosis, loss of correction, and revision surgery). The training dataset may further include biomechanical simulation data generated from finite element models or in vitro testing, which may augment or supplement the clinical data. The machine learning models may be trained using supervised learning techniques, in which the model learns to map the input patient-specific data to the output spinal rod configuration and predicted outcomes based on labeled examples in the training dataset. For example, the model may be trained to minimize a loss function that penalizes deviations between the model's predicted rod configuration and the rod configuration that was actually used for a given patient, weighted by the quality of the postoperative outcome achieved. In this manner, the model may learn to preferentially recommend rod configurations that are associated with favorable postoperative outcomes and low complication rates across the training dataset.

In one embodiment, the one or more machine learning models may be further trained or refined using unsupervised learning or semi-supervised learning techniques. For example, the system 1000 may apply clustering algorithms to identify patterns or subgroups within the historical patient data that share similar anatomical characteristics, deformity types, or biomechanical profiles, and may use the identified patterns to improve the accuracy and specificity of the rod configuration recommendations for patients falling within each subgroup. In another embodiment, the machine learning models may be trained using reinforcement learning, in which the model iteratively refines its rod configuration recommendations based on feedback signals derived from postoperative outcomes, biomechanical simulations, or surgeon evaluations.

Once trained, the one or more machine learning models may be deployed within the system 1000 and stored on the non-transitory computer-readable medium for execution by the processor 500. During use, the system 1000 may receive the patient-specific inputs as described herein, preprocess the inputs (e.g., normalizing values, extracting features from imaging data, registering anatomical landmarks), and provide the preprocessed inputs to the trained machine learning model. The trained model may generate a recommended spinal rod configuration and an associated failure-state risk percentage, which may be displayed to the surgeon on screen 300 or another output device. In an embodiment, the trained model may generate a plurality of recommended spinal rod configurations, each with an associated failure-state risk percentage and predicted postoperative outcome, allowing the surgeon to evaluate and compare the options as described herein. The system 1000 may further allow the surgeon to adjust one or more input parameters and observe the corresponding changes to the model's recommended rod configuration and failure-state risk percentage in real-time or near real-time, enabling an iterative and interactive planning process.

In some embodiments, the one or more machine learning models may be updated or retrained over time as new patient data and postoperative outcome data become available. For example, the system 1000 may collect postoperative outcome data for patients treated using the system's recommended rod configurations, and may use the collected data to retrain or fine-tune the machine learning models, thereby improving the accuracy and reliability of the system's recommendations over time. The retraining may be performed periodically (e.g., after a predetermined number of new cases), continuously, or on-demand. In an embodiment, the retraining may be performed using federated learning techniques, in which the machine learning models are trained across multiple institutions or devices within the system 1000 without sharing raw patient data, thereby preserving patient privacy while enabling the models to learn from a broader and more diverse training dataset.

It is to be understood that the computational approaches described herein, including finite element analysis, rule-based optimization, machine learning (e.g., neural networks, random forests, support vector machines, gradient boosting models), supervised learning, unsupervised learning, semi-supervised learning, reinforcement learning, and federated learning, are provided as non-limiting examples of computational methodologies that may be utilized by the system 1000 to determine the spinal rod configuration, generate the curvature template, calculate the failure-state risk percentage, and provide output instructions as described herein. The system 1000 is not limited to any particular computational approach, and may utilize any suitable computational methodology, algorithm, model, technique, or combination thereof, whether now known or hereafter developed, that is capable of receiving the patient-specific inputs described herein and generating the outputs described herein.

For example, the system 1000 may additionally or alternatively utilize statistical regression models, Bayesian inference, genetic algorithms, evolutionary optimization, physics-informed neural networks, digital twin modeling, or any other suitable analytical or predictive methodology, without departing from the scope of the present disclosure. Further, the system 1000 may utilize any combination of the foregoing computational approaches, either in series or in parallel, to determine the spinal rod configuration and associated outputs. The specific computational approach or combination of approaches employed by the system 1000 may be selected based on the available data, the desired accuracy, the computational resources of the system 1000, the clinical context, or surgeon preference, and may vary from patient to patient or from procedure to procedure.

Additionally, with the disclosed spinal rods, systems, and methods, once the preoperative plan has been made, the screw placement locations can allow a surgeon to visualize the curvature of the potential spinal rod to be placed. Based on the screw placement locations, the patient's anatomy, and the defined linear segments, tapering thereof (e.g., Bezier zones), and other rod parameters, the system 1000 may generate a curvature template for the spinal rod. The curvature template may represent the predicted three-dimensional shape and stiffness profile of the spinal rod as it would be contoured and implanted in the patient, and may account for the defined linear segment diameters, the tapering (e.g., Bezier zones) between adjacent segments, the overall curvature of the rod, and the rod material or materials.

The curvature template may further allow for prediction of the unloading of the spinal rod when locked in an anchor, screw, or the like. For example, the system 1000 may calculate or estimate the forces that would be exerted at each anchor point along the spinal rod, including the forces at the UIV and the proximal screws, based on the curvature template, the rod stiffness profile, the patient's bone quality, the patient's spinal alignment, and the corrections applied during the preoperative and intraoperative planning. The predicted unloading forces may account for the contribution of the tapered segments (e.g., Bezier zones) to the stiffness transition along the rod, and may reflect the more gradual force distribution provided by the Bezier-defined taper as compared to a conventional stepped or constant-diameter rod.

With the curvature template, the system 1000 may generate a failure-state risk percentage that translates the predicted unloading forces into a quantified risk of a failure state. The failure-state risk percentage may account for one or more factors including, without limitation, the predicted forces at the UIV and proximal screws, the patient's bone mineral density and endplate strength, the rod stiffness profile and material, the interbody cage parameters (if applicable), the degree of correction achieved, and the patient's overall spinal alignment parameters such as pelvic incidence, sagittal vertical axis, and T1 pelvic angle. The failure-state risk percentage may reflect the risk of one or more failure modes including, without limitation, PJK, PJF, adjacent segment disease, rod fracture, screw loosening, cage subsidence, pseudarthrosis, and loss of correction. The failure-state risk percentage may be displayed to the surgeon on screen 300 or another output device, allowing the surgeon to make an informed decision on further surgical pathways to address or mitigate the predicted risk. In an embodiment, the surgeon may manipulate one or more rod parameters, cage parameters, or screw placement locations within the system 1000 and observe the corresponding changes to the curvature template and the failure-state risk percentage in real-time or near real-time, thereby enabling an iterative evaluation of different construct configurations to identify an optimal surgical plan.

The algorithm utilized by the system 1000 may select or optimize the Bezier curve parameters for the tapered segments of the spinal rod. Specifically, the system 1000 may determine the number of control points, their positions, and the order of the Bezier curve based on the patient's imaging data, bone mineral density, spinal alignment, and desired stiffness profile. The selection of the Bezier curve parameters may be coordinated with the curvature template and the failure-state risk percentage such that the tapered segments are optimized to achieve the desired stiffness transition while minimizing the predicted failure-state risk.

For example, the patient may provide inputs to device 100; the surgeon may provide a preoperative assessment to device 400; the surgeon may provide an intraoperative assessment through device 200; processor 500 may store inputs made at different times, by different people, and over different devices received through the communication network or framework; processor 500 may, at the time the surgeon desires an output, e.g., during surgery, aggregate and analyze the inputs with an algorithm or using the criteria or standards described herein to provide an output for possible in-situ adjustments to the spinal rods or implantation of the spinal rods thereof based on patient anatomy and desired results; and the like.

In an embodiment, the system may provide output instructions for rod contouring, such as bending or shaping of the spinal rod, where the surgeon can manually bend or contour the rod using rod benders to better match the patient’s anatomy, such as the patient’s sagittal and coronal alignment. Patient-specific contouring can help maintain physiological spinal curvature while reducing undue stress at junctional segments whereas over-bending or under-bending can lead to excessive strain or misalignment, increasing the risk of mechanical failure. In an embodiment, the system may similarly provide output instructions for rod rotation, where rotation of the rod, for example, after being secured into the pedicle screws, can align the spine in the axial plane, improving overall spinal balance and desired contact with the spinal column.

In an embodiment, the system may provide output instructions for sequential compression and distraction of the spinal rod relative the spinal column using rod holders, compressors, or distractors, to fine-tune spinal height and alignment. For example, compression can enhance fusion by increasing contact between vertebral surfaces, whereas distraction can help restore disc height or correct kyphotic deformities. In an embodiment, the system may provide output instructions for rod translation including adjusting the position of the rod relative the screws for improved alignment and load distribution. Such adjustments can assist in ensuring forces are evenly distributed, preventing point-loading and excessive stress at specific segments.

In an embodiment, the system may provide output instructions for rod stiffness modulation, for example, selecting different materials having different flexibility and rigidity for all or a particular portion of the spinal rod. For example, the output instructions could recommend using a more flexible titanium rod at the proximal end and a stiffer cobalt-chromium rod distally to manage stress distribution and reduce the risk of PJK. In an embodiment, the system may provide output instructions for cross-link placement where adding or adjusting cross-links can add stability, reduce torsional forces, and improve load sharing between the bilateral rods. For example, the cross-links can be repositioned intraoperatively to optimize support based on the patient’s anatomy and mechanical needs. Similarly, the system may provide output instructions for screw type and placement where adding or adjusting screw types and placement can enhance construct stability by reducing torsional forces and improving load sharing along and across the spinal column. As with cross-links, the screws can be repositioned intraoperatively to optimize support based on the patient’s anatomy and mechanical needs.

In an embodiment, the system may provide output instructions for rod cutting to a desired length and rod end modification. For example, rods may be provided to a surgeon at a length that is longer than needed so the rod can be trimmed by the surgeon to the exact dimensions desired for a particular patient. Additionally, smoothing or tapering the cut end can minimize stress concentrations and potential implant failure.

As described herein, the disclosed spinal rods, systems, and methods may facilitate in-situ adjustments to address current anatomy as well as to prevent future disease and complications. The disclosed spinal rods, systems, and methods may show improved longevity and comfort to the patient over time without need for revisions. The disclosed spinal rods, systems, and methods may prioritize customization to a patient's anatomy through several different adjustments of the spinal rod itself and the implantation of the spinal rod thereof into a patient to unload forces at the UIV proximal segment and to account for and match bilateral forces across and along the spinal column. The disclosed spinal rods, systems, and methods may develop, provide, and/or analyze inputted information to output instructions to the surgeon during operation to provide the improved spinal rods, systems, and methods to the patient.

In summary, the present disclosure provides a spinal rod comprising a plurality of linear segments and at least one tapered segment that together provide variable stiffness along the longitudinal axis, with segment parameters configurable to a patient's anatomy. The present disclosure further provides a system comprising a processor and a non-transitory computer-readable medium that receives preoperative and intraoperative inputs, determines a patient-specific spinal rod configuration, generates a curvature template, and provides output instructions for in-situ adjustment or implantation. The present disclosure further provides a method of implanting a spinal rod that includes preoperative and intraoperative assessments, computational analysis, in-situ adjustments, and implantation. These three aspects of the disclosure, the spinal rod, the system, and the method, may operate independently or in coordination with one another to achieve a patient-specific spinal construct that maintains alignment, distributes forces evenly across the construct, accounts for bilateral forces, and reduces the risk of complications such as stress shielding, Proximal Junctional Kyphosis (PJK), Proximal Junctional Failure (PJF), and Adjacent Segment Disease.

Although the embodiments of the present teachings have been illustrated in the accompanying drawings and described in the foregoing detailed description, it is to be understood that the present teachings are not to be limited to just the embodiments disclosed, but that the present teachings described herein are capable of numerous rearrangements, modifications and substitutions without departing from the scope of the claims hereafter. The claims as follows are intended to include all modifications and alterations insofar as they come within the scope of the claims or the equivalent thereof.

What is claimed is:

1. A spinal rod for spinal surgery, comprising:
a plurality of linear segments arranged along a longitudinal axis of the spinal rod, the plurality of linear segments comprising at least a first linear segment having a first diameter and a second linear segment having a second diameter different from the first diameter; and
at least one tapered segment disposed between adjacent linear segments of the plurality of linear segments, the at least one tapered segment configured to provide a gradual transition in diameter between the adjacent linear segments,
wherein the plurality of linear segments and the at least one tapered segment are configured to provide a variable stiffness along the longitudinal axis of the spinal rod,
wherein the at least one tapered segment is configured to distribute force across a surface of the spinal rod, and
wherein a length, a diameter, a tapering, a location, and a number of the plurality of linear segments and the at least one tapered segment are capable of being configured to a patient's anatomy.

2. The spinal rod of claim 1, wherein the first linear segment and the second linear segment are adjacent linear segments, and
wherein the at least one tapered segment is disposed between the first linear segment and the second linear segment.

3. The spinal rod of claim 1, wherein the at least one tapered segment comprises a Bezier tapered segment.

4. The spinal rod of claim 1, wherein a proximal portion of the spinal rod comprises a first material and a distal portion of the spinal rod comprises a second material different from the first material, and wherein the first material is more flexible than the second material.

5. The spinal rod of claim 1, further comprising at least one curved segment disposed along the longitudinal axis of the spinal rod.

6. The spinal rod of claim 1, wherein the plurality of linear segments comprise at least three linear segments having different lengths, and wherein the at least three linear segments are arranged in a predetermined order along the longitudinal axis based on the patient's anatomy.

7. The spinal rod of claim 1, wherein the spinal rod has a curvature configured to follow a natural sagittal curvature of the patient’s anatomy.

8. A system for determining a configuration and placement of a spinal rod in a patient, the system comprising:
a processor; and
a non-transitory computer-readable medium configured to store instructions that, when executed by the processor, cause the system to:
receive one or more inputs related to a preoperative assessment of the patient and an intraoperative assessment of the patient, the one or more inputs comprising at least one of imaging data, physician observations, diagnoses, or anatomical dimensions of the patient;
determine, based on the one or more inputs, a spinal rod configuration comprising a plurality of linear segments and at least one tapered segment configured to provide variable stiffness, the spinal rod configuration being based on the patient's spinal alignment, bone quality, and anatomical dimensions;
generate a curvature template based on the spinal rod configuration; and
provide instructions for at least one of in-situ adjustment or implantation of the spinal rod based on the spinal rod configuration and the curvature template.

9. The system of claim 8, wherein the spinal rod configuration is determined based on stress distribution and load transfer associated with the patient's spine.

10. The system of claim 8, wherein the curvature template comprises a failure-state risk percentage.

11. The system of claim 8, wherein the one or more inputs further comprise at least one of MRI data, CT data, DEXA scan data, osteotomy angles, sagittal balance, pelvic incidence, or interbody cage angles.

12. The system of claim 8, wherein the instructions further cause the system to analyze the one or more inputs using at least one of computational modeling, artificial intelligence, or intraoperative navigation.

13. The system of claim 8, wherein the instructions comprise one or more instructions of rod contouring, rod rotation, sequential compression and distraction, rod translation, rod stiffness modulation, cross-link placement, screw type and placement selection, or rod cutting and end modification.

14. The system of claim 8, wherein the instructions further cause the system to determine a first spinal rod configuration for a first side of the patient's spinal column and a second spinal rod configuration for a second side of the patient's spinal column, the first spinal rod configuration and the second spinal rod configuration being configured to provide symmetric load transmission across the spinal column.

15. A method of implanting a spinal rod in a patient, comprising:
performing a preoperative assessment on the patient;
performing an intraoperative assessment on the patient;
providing one or more inputs related to the preoperative assessment and the intraoperative assessment into a computational system;
analyzing, by the computational system, the one or more inputs to generate output instructions for implantation of the spinal rod, the spinal rod comprising a plurality of linear segments and at least one tapered segment;
performing, based on the output instructions, at least one adjustment to the spinal rod; and
implanting the spinal rod in the patient.

16. The method of claim 15, wherein the at least one adjustment comprises one or more of rod contouring, rod rotation, sequential compression and distraction, rod translation, rod stiffness modulation, cross-link placement, screw type and placement selection, or rod cutting and end modification.

17. The method of claim 15, further comprising implanting a second spinal rod on an opposite side of the patient's spinal column, wherein the spinal rod and the second spinal rod are configured to provide symmetric load transmission across the spinal column.

18. The method of claim 15, wherein the one or more inputs comprise at least one of imaging data, physician observations, diagnoses, or anatomical dimensions of the patient.

19. The method of claim 15, wherein the computational system generates a curvature template based on a spinal rod configuration, the curvature template comprising a failure-state risk percentage.

20. The method of claim 15, wherein the plurality of linear segments and the at least one tapered segment are configured to provide variable stiffness along a length of the spinal rod.
